(19) United States
(12) Patent Application Publication
(10) Pub. No.: US 2022/0079548 A1
(43) Pub. Date: Mar. 17, 2022

(54) ELECTRONIC STETHOSCOPE WITH VOLUME ADJUSTMENT

(71) Applicant: 3M INNOVATIVE PROPERTIES COMPANY, St. Paul, MN (US)

(72) Inventor: William Bedingham, Woodbury, MN (US)

(21) Appl. No.: 17/420,128

(22) PCT Filed: Jan. 6, 2020
(86) PCT No.: PCT/IB2020/050051

§ 371 Date: Jun. 30, 2021

Related U.S. Application Data
(60) Provisional application No. 62/790,694, filed on Jan. 10, 2019.

(51) Int. Cl. A61B 7/04; A61B 7/00

(52) U.S. Cl. CPC A61B 7/04; A61B 7/003

(57) ABSTRACT

Aspects of the present disclosure relate to a stethoscope that includes a chestpiece having an inside surface and an outside surface. A portion of the inside surface forms a bell and a portion of the outside surface is electrically conductive. The stethoscope includes a plurality of sensors and a speaker that communicatively coupled to a first sensor. The stethoscope also includes a controller circuit that is configured to receive a plurality of sensor readings from the plurality of sensors. The controller circuit can determine a noise profile based on the plurality of sensor readings and a first volume output through the speaker, determine whether a noise profile threshold is met by the noise profile; and reduce volume output through the speaker from the first volume to a second volume based on the noise profile threshold being met.

### BACKGROUND

Mechanical stethoscopes have been developed to detect sounds produced by the body, such as heart and lung sounds. The stethoscope, for example, is a fundamental tool used in the diagnosis of diseases and conditions of the cardiovascular system. It serves as the most commonly employed technique for diagnosis of such diseases and conditions in primary health care and in circumstances where sophisticated medical equipment is not available, such as in remote areas.

Users readily appreciate that detecting relevant cardiac symptoms and forming a diagnosis based on sounds heard through the stethoscope. Electronic stethoscopes have the ability to amplify certain auscultation sounds. Electronic stethoscopes have the potential to provide the clinical user with both amplified and enhanced sound.

### SUMMARY

With current electronic stethoscopes, the increased sound amplification can also be associated with harsh and unacceptable audio noise to the listener that is caused by a number of factors, for example, moving/sliding the electronic stethoscope over patient's body to locate patient sound signal, adjusting the user's grip on the chest piece, tremor/shaking of the user's arm/hand holding the stethoscope, motion/breathing of patient, or accidental bumping of the chestpiece.

Aspects of the present disclosure relate to a stethoscope that includes a chestpiece having an inside surface and an outside surface. A portion of the inside surface forms a bell and a portion of the outside surface is electrically conductive. The stethoscope includes a plurality of sensors and a speaker that communicatively coupled to a first sensor. The stethoscope also includes a controller circuit that is configured to receive a plurality of sensor readings from the plurality of sensors. The controller circuit can determine a noise profile based on the plurality of sensor readings and a first volume output through the speaker, determine whether a noise profile threshold is met by the noise profile; and reduce volume output through the speaker from the first volume to a second volume based on the noise profile threshold being met.

Additional aspects of the present disclosure also relate to a method of reducing the volume of an electronic stethoscope in response to a plurality of sensor readings.

### BRIEF DESCRIPTION OF DRAWINGS

FIG. 1 illustrates a stethoscope, according to aspects of the present disclosure.

FIG. 2 illustrates a side view of a chestpiece, according to aspects of the present disclosure.

FIG. 3 illustrates a top view of the chestpiece of FIG. 2, according to aspects of the present disclosure.

FIG. 4 illustrates a flowchart of a method for using a noise profile, according to aspects of the present disclosure.

FIG. 5 illustrates a flowchart of a method for analyzing motion signals useful in generating the noise profile, according to aspects of the present disclosure.

FIG. 6 illustrates a flowchart of a method for analyzing capacitance signals useful in generating the noise profile, according to aspects of the present disclosure.

FIG. 7 illustrates a flowchart of a method for analyzing sounds useful in generating the noise profile, according to aspects of the present disclosure.

FIG. 8 illustrates a flowchart of a method for aggregating one or more sensor readings to generate the noise profile, according to aspects of the present disclosure.

FIG. 9 illustrates a block diagram of controller circuit useful in performing aspects of the present disclosure.

### DETAILED DESCRIPTION

Aspects of the present disclosure relate to an electronic stethoscope that can reduce a volume from a first level to a second level based on a plurality of sensor readings and returning the volume to the first level. In at least one embodiment, the sensor readings are generated by interaction with the user (excluding any preset user settings) and can indicate instability with respect to the user's hand. A user can be a clinician (such as a doctor, nurse, or medical worker) or the user can be a patient that uses the electronic stethoscope on himself/herself (e.g., as part of a take home study).

For example, the electronic stethoscope can use information from the electronic sound sensor (e.g. Piezo crystal), a touch sensor on the stainless-steel chest piece shell (e.g. capacitance sensor), and a motion sensor (e.g. 3-axis accelerometer and/or gyroscope) to adjust and optimize the sound level of the stethoscope during measurement. During motion or changes in the user's grip, the audio volume can be markedly reduced or muted. Once the stethoscope has been positioned and stabilized over an auscultation site, the audio volume is slowly ramped up. The user can adjust the positioning to optimize the patient signal and the audio volume will automatically adjust to prevent harsh sounds from being heard.

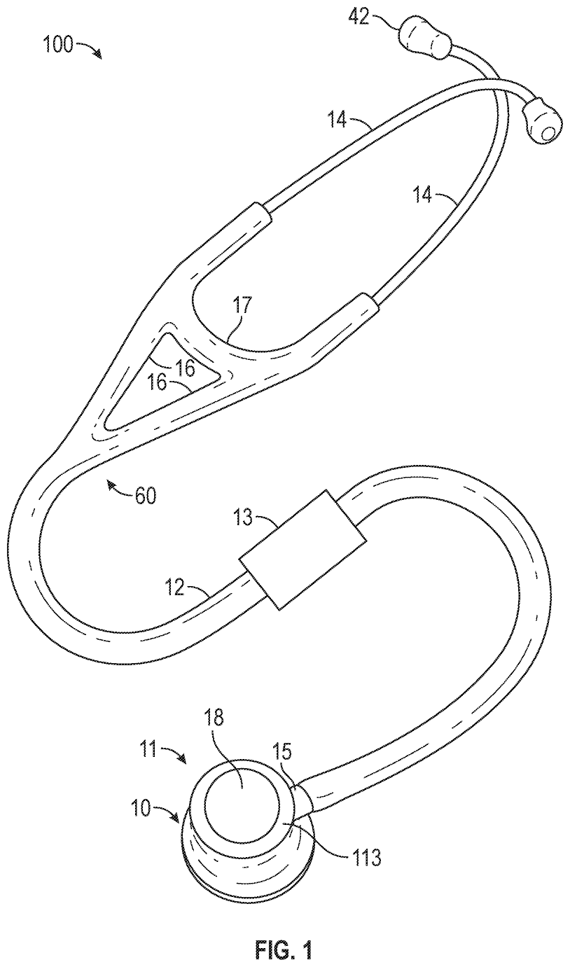

FIG. 1 illustrates a stethoscope 100 comprising a stethoscope chestpiece 10 which further comprises body member 11 formed of metallic and/or thermoplastic compositions. Stethoscope chestpiece 10 is shown attached to a conventional headset such as those commercially available under the trade designation Littman by 3M (St. Paul, Minn.) which comprises elongated flexible tubing 12 between stethoscope chestpiece 10 and ear tubes 14. In the lower end of flexible tubing 12 which attaches to stethoscope chestpiece 10, passages can be adapted to be coupled to stem fitting 15 of [symbol] pe chestpiece 10. The upper end of flexible tubing 12 bifurcates into coupling arms 16, each [symbol] attaches to one of the ear tubes 14 and each of which contains one of the ear tips 42. Ear tubes 14 are secured together by tubing 17. The flexible tubing 12 and the ear tubes 14 can be optional, for example, the stethoscope chestpiece 10 can communicatively pair with wireless headsets such as Bluetooth® head phones. In at least one embodiment, a wireless headset can be coupled with the flexible tubing 12 and ear tubes 14.

In at least one embodiment, the flexible tubing 12 can include a yoke 60 that forms a y-shape or t shape within the tubing 12. In at least one embodiment, the stethoscope chestpiece 10 comprises one or more electronic components as described further herein. Thus, the stethoscope 100 can be an electronic stethoscope similar to that sold by 3M under the trade designation Littmann. The electronic stethoscope 100 can selectively amplify auscultation sounds or body signals via sound output through a speaker. The ear tips 42 can be fluidically coupled to the speaker described herein through the yoke 60 and tubing 12.

In at least one embodiment, at least some of the electronic components can be housed in a housing 13. The electronic components can include a speaker, controls, a battery, one or more processors, touch sensors, voltage converter, display. The electronic components within the housing 13 can be electrically coupled to electronic components of the stethoscope chestpiece 10 (such as sound sensors).

In at least one embodiment, the stethoscope chestpiece 10 can be configured to resemble a traditional stethoscope such as the model Cardiology IV under the trade designation Littmann by 3M (St. Paul, Minn.). Such a configuration may allow for ease of adoption for electronic stethoscopes. In at least one embodiment, the chestpiece 10 can include a display 18. The display 18 can enable both visual cues of different functions of the stethoscope and or control functions of the stethoscope 100. The display 18 may also provide user touch input. In at least one embodiment, the visualizations can include a phonocardiograph of different auscultation sounds. As shown herein, the display 18 can include a border 113 proximate to an edge of the body member 11. In some embodiments, the display 18 can extend to the edge of the chestpiece to form a streamlined appearance.

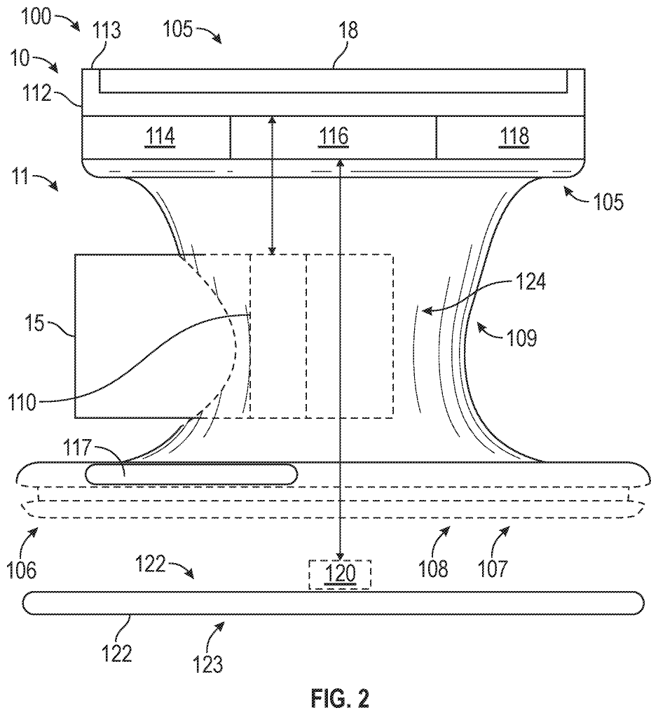

FIG. 2 illustrates a side cross-sectional view of the stethoscope 100 including the stethoscope chestpiece 10.

The stethoscope chestpiece 10 can have a body member 11. In at least one embodiment, the body member 11 can be at least partially metallic such that it is electrically conductive along a portion therein. In at least one embodiment, a portion of the body member 11 can form a battery by using two different materials. At least a portion of the body member 11 (preferably the entire body member) can be formed of stainless-steel. A stainless-steel shell can provide a conductive surface for the capacitive [symbol] sor, electrical shielding of the internal sensors and electronics, and can also be polished to [symbol] unwanted friction noise that is caused by the user adjusting their grip.

In at least one embodiment, the body member 11 can have a top portion 105 and a bottom portion 106. As shown, the display 18 and electronic components can be positioned on the top portion 105 and a diaphragm 122 contacts the bottom portion 106.

In at least one embodiment, the electronic components can be placed proximate to the top portion 105 opposite the bottom portion 106. In at least one embodiment, the electronic components can be positioned such that they are located coplanar with a single printed circuit board. For example, the chestpiece 10 can include a controller circuit 112 which comprises one or more processors and a memory. The controller circuit can include one or more computer processors communicatively coupled to a memory which can include instructions that when executed by the one or more computer processors cause the one or more computer processors to perform various operations described further herein. The controller circuit 112 can be electrically coupled to the display 18, the audio circuit 114, the motion sensor 116, the capacitance sensor 118, the speaker 110, and the sound sensor 120. Speaker 110 can be located in a different portion of the stethoscope (e.g. near headset or within the housing 13 in FIG. 1). The sound can also be transmitted wirelessly to a headset (e.g., ear tubes, or a wireless headset). Although not pictured, the controller circuit 112 can also be electrically coupled to a power source (e.g., a battery) that may be housed internally within a cavity of the body member 11.

In at least one embodiment, the audio circuit 114 is configured to receive the electrical signal from the sound sensor 120, perform one or more functions, and output an auscultation sound through the speaker 110. Various signal processing can be performed, e.g., such as that described in WO 2002032313A2, or WO 2013086112A1.

The sound sensor 120 can transmit the analog signal through the audio circuit 114. The audio circuit 114 can include a pre-emphasis filter, one or more bandpass filters, analog-to-digital converter, and digital-to-analog converter. The audio circuit 114 can perform signal processing on the auscultation sound from the first sensors to produce a processed signal.

For example, the audio circuit 114 can have various electronic filters for each mode to selectively enhance the stethoscope sounds (e.g., produce a processed signal). For example, a bell mode can amplify sounds from 20 to 1000 Hz but emphasize lower frequency sounds between 20 to 200 Hz. A diaphragm mode can amplify sounds from 20 to 2000 Hz but emphasize sounds between 100 to 500 Hz. An extended range mode can amplify sounds from 20 to 2000 Hz similar to a diaphragm mode but provide more low frequency response between 50 to 500 Hz. [symbol] n at least one embodiment, the audio circuit 114 can play the processed signal through the [symbol] 10 at a first (initial, preset) volume. The volume can refer to the degree of loudness or intensity of a sound. The stem fitting 15 (having an internal surface of a wall and an exterior surface of the wall) and can also be modified to receive electronic components. For example, a speaker 110 or sound sensor 120 can be placed within the internal surface of the wall of the stem fitting 15 such that sound is amplified from the sound sensor and output via a tubing. For example, the speaker 110 can be fluidically coupled to eartips, e.g., such that the speaker 110 can broadcast sound through the tubing 12. In at least one embodiment, the stem fitting 15 is generally configured to have an opening proximate (e.g., near but not as close as adjacent or bordering), or adjacent to the speaker 110. In at least one embodiment, the speaker 110 or sound sensor 120 can be present within the tubing 12.

In at least one embodiment, a motion sensor 116 can be any device that senses motion of the chestpiece 10. The motion sensor 116 can be based on acceleration of the chestpiece 10, the visual motion relative to a fixed surface, or combinations thereof. For example, the motion sensor 116 can be an accelerometer such as a 2-axis, or 3-axis accelerometer. The motion sensor 116 can be proximate to the chestpiece 10, contained within the chestpiece 10, or adjacent to the chestpiece 10 such that motion of the chestpiece 10 in a two-dimensional or three-dimensional space (relative to a portion of a patient and/or user) is obtained. For example, the motion sensor 116 can capture the motion relative to a chest or back portion of a patient. In another example, the motion sensor 116 can capture motion relative to how the chestpiece 10 is manipulated by a user, such as the motion of the user's hand. In addition to a motion sensor, an optical, or electrical (e.g. electromagnetic, capacitive, acoustic) proximity sensor 117 can detect when the stethoscope chestpiece 10 is near or touching the patient's body.

In at least one embodiment, a motion sensor 116 can detect subtle motions that occur during auscultation (e.g. user/patient tremor, user changing grip pressure on chest piece) and the controller circuit 112 can subtract this noise signal from the audio signal (active noise cancellation). The motion sensor 116 can also monitor the orientation of the patient during auscultation (e.g. sitting/standing, lying down) and the controller circuit 112 can adjust the audio volume and motion sensitivity appropriately. The motion sensor 116 can have multiple sensitivity levels. For example, sensitivity can be increased when the chestpiece 10 is in contact with the patient (as indicated by limited z-axis motion and moderate x and y axis motion), and the sensitivity can be decreased when the chestpiece 10 is being moved which can allow for the detection of subtle motions.

In at least one embodiment, the diaphragm 122 can have a sound sensor 120 attached thereon. The sound sensor 120 can also be present in another part of the stethoscope chestpiece 10 such as proximate to the stem fitting 15. The sound sensor 120 can receive vibrations from a diaphragm 122 and translate the vibrations into electrical signals. The sound sensor 120 be a piezoelectric [symbol] ne or any transducing or contact microphone configured to receive sounds. A piezoelectric [symbol] ne can differ from a microphone in that a piezoelectric microphone does not have a built-in diaphragm. A piezoelectric microphone can replicate the signal of the underlying substrate (such as a stethoscope diaphragm 122. In at least one embodiment, the piezoelectric microphone can be laminated directly to the diaphragm 122 and secured with a flexible adhesive (generally having a modulus of elasticity less than 100000 psi or a modulus of elasticity no greater than the diaphragm 122 at 20 C). According to at least one embodiment, the piezoelectric sensor can avoid the use of foam disposed proximate thereof.

The sound vibration properties of the diaphragm 122 can be optimized to respond to body sounds of interest (e.g. heart, 50-700 Hz, lungs, 100-1500 Hz). For example, changing the diaphragm 122 thickness, stiffness, and supporting elastomeric ring properties will alter the natural harmonic frequency of the mechanical system. Electronic and software filtering can further enhance the resultant sound.

In at least one embodiment, the sound sensor 120 can also be used to detect and eliminate harsh sounds to the listener. For example, during normal auscultation, the “power” of the sound signal is periodic, low magnitude, and within specific frequency ranges. But during motion, the sound is continuous and contains significantly enhanced power. The power signal can be used to adjust and optimize the audio volume. The controller circuit 112 can be configured to increase audio volume when motion stable and sound power is low, or reduce or mute audio volume when sound volume exceeds a threshold value as described herein.

In at least one embodiment, the chestpiece 10 can have an inside surface 107 which includes a bell 108 which forms a cavity with the diaphragm 122. The diaphragm 122 can have a first side 123 that faces the patient and a second side 122 that faces the inside surface 107 of the chestpiece 10.

In at least one embodiment, the chestpiece 10 can have an outside surface 109, a portion of which may be electrically conductive. The chestpiece 10 can be primarily formed from a metallic composition which may be electrically conductive. For example, if the chestpiece 10 is investment-cast brass, then the chestpiece 10 can be inherently conductive. In at least one embodiment, a portion of the chestpiece 10 can be polymeric, including metal-polymer compositions that are electrically conductive. The polymer can also contain carbon or graphite. The chestpiece 10 can also have electrically conductive sections forming a portion of the chestpiece 10, e.g., a hand grip portion 124 where a user can grasp the chestpiece 10 with two or more fingers. The electrically conductive portion, e.g., hand grip portion 124, can be electrically coupled to the capacitance sensor 118. In at least one embodiment, the electrically conductive portion is distinct from an electrode sensor. For example, an electrode sensor may be used to measure electrical signals from a patient's body. The [symbol] ly conductive portion can be used to measure capacitance of a portion of the user's body. In [symbol] ne embodiment, the capacitance is the ability of the conductive portion to store an electrical charge from the user. The conductive portion of the chestpiece 10 can also be covered with a thin non-conductive material (e.g. polymeric material) so that the user does not directly touch the conductive materials.

The diaphragm 122 may also be electrically coupled to the capacitance sensor 118 to sense when the chestpiece is in contact with the patient's body in addition to the capacitance sensor 118 being in contact with the clinician's hand. In at least one embodiment, the chestpiece 10 can include one or more electrode sensors disposed on the bottom portion 106, proximate to the diaphragm 122.

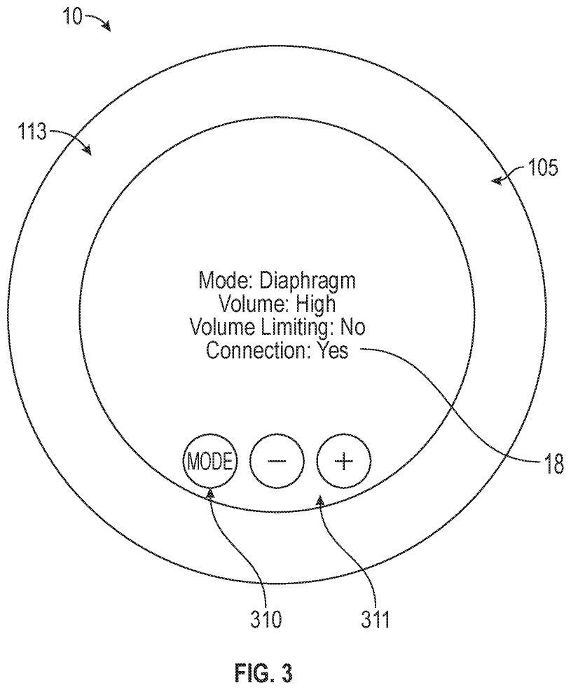

FIG. 3 illustrates a top elevational view of the chestpiece 10. The top portion 105 can have a display 18 disposed thereon as discussed herein. The display 18 can be surrounded by a border 113. The display 18 can visually communicate status to a user, for example, various operations that the stethoscope can perform. For example, the display 18 can communicate the mode of operation of the stethoscope.

The volume can also be indicated by the display 18 to alert a user for a preset volume level based on user preferences. A volume limiting option, described herein, can be used to lower the volume from the preset volume level based on sensor readings from the one or more sensors. A connection option can be used to indicate the presence of Bluetooth® or other wireless connection.

Control of the stethoscope can be provided by physical buttons or via a touch sensitive screen. For example, an icon 310 featuring menu options can be used to cycle through different modes while icons 312 featuring volume controls 311 can be used to adjust the preset volume. While the volume limiting option uses one or more sensor readings to limit the preset volume, the one or more sensor readings do not include manual adjustments to the volume by the user. For example, the user adjusting the preset volume level via buttons or capacitive touch manually.

The user interface may also be located in a different section of the stethoscope such as the housing 13 in FIG. 1. The location can allow the user to use one hand to place and maneuver the chestpiece on the patient, while the other hand can adjust the volume and/or filter settings without disturbing the chestpiece 10. Non-mechanical touch sensors can be used to minimize vibration in the stethoscope 100 that can be picked up as unwanted sound or changes the motion sensor 116 in the chestpiece 10.

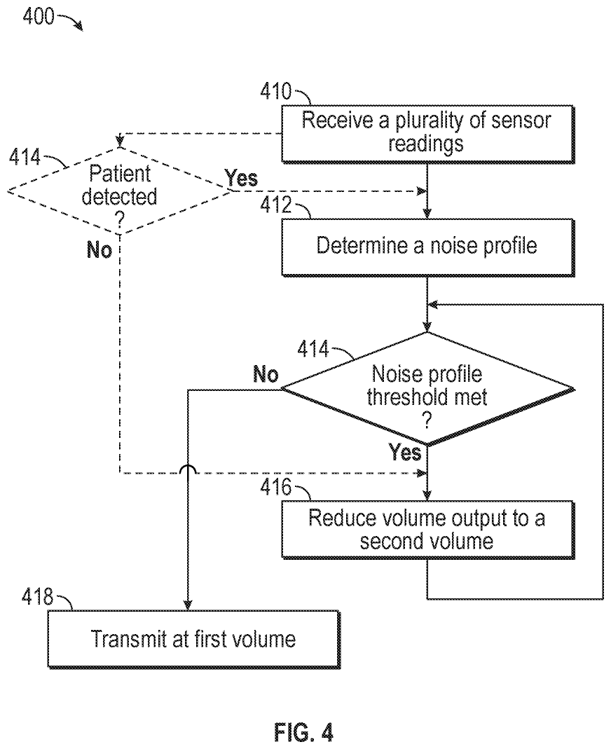

FIG. 4 illustrates a flowchart of a method 400 for modifying the volume of the stethoscope. The method 400 can be performed by a processor and memory of the controller circuit and begin at block 410. [symbol] n block 410, the controller circuit can receive a plurality of sensor readings from the plurality [symbol] s. Each sensor can receive a sensor reading for a particular sensor type. For example, a motion sensor can receive motion of a portion of the stethoscope (e.g., a body member) such as that relative to the diaphragm. The plurality of sensors does not include a button or capacitive touch control used to control a preset volume level of the stethoscope. For example, a user can use one or more buttons to control the preset volume level manually. An aspect of the present disclosure is the automatic adjustment by the controller circuit based on a noise profile that can indicate chestpiece instability.

In at least one embodiment, at least one of the plurality of sensor readings can be from a proximity sensor that senses the presence of a patient. For example, the controller circuit can first determine if the proximity sensor indicates the presence of the patient before block 412. If the proximity sensor does not detect the patient, then the controller circuit can automatically reduce the volume.

In block 412, the controller circuit can determine a noise profile based on one or a plurality of sensor readings and a first volume output through the speaker. The first volume (or preset volume) can be an indication of a user's preferences regarding the volume level that is output through the speaker onto the eartips. For example, if the volume is preset at a level of 9 out of 10, then the preset volume level can be indicative of preferences of the user so that the user can hear auscultation sounds.

In at least one embodiment, the noise profile can represent noise present from the stethoscope. For example, a dragging of the stethoscope across clothing of a patient can produce unwanted noise including high decibel pressure volumes and/or nonbiological frequencies that the user may not want amplified. In another example, an unstable chestpiece may amplify sounds that a user may not want amplified. In another example, an unstable user grip may also amplify sounds that a user may not want to be amplified.

In at least one embodiment, the noise profile can be an aggregate of one or more scores determined by the controller circuit for each sensor. The score is a numeric value based on how much undesired signal (e.g., noise) is indicated by the sensors. The noise profile can account for various weights for each sensor score. The noise profile can be described further herein. Alternatively, each sensor may have its own response threshold and the ability to independently mute or reduce the sound volume.

In block 414, the controller circuit can determine whether a noise profile threshold is met by the noise profile. In at least one embodiment, the noise profile threshold can be representative of the likelihood of amplifying noise or an undesirable signal. For example, if the noise profile threshold is met, then there may be a high likelihood of amplifying noise for the user. In at least one embodiment, [symbol] oller circuit can determine whether the noise profile threshold is met by comparing the noise [symbol] d the noise profile threshold.

In block 416, the controller circuit can reduce the volume output through the speaker from the first volume to a second volume based on the noise profile threshold being met. Thus, a duration of the second volume occurs for as long as the noise profile threshold is met and likely undesirable sounds from the sound sensor can be reduced. In at least one embodiment, the time period to reduce the volume output can be near instantaneous. For example, a reduction in the volume output can occur no greater than 50 ms of a sound being received by the sound sensor. In at least one embodiment, the second volume can be from 0% to 99%, 0% to 90%, 0% to 80%, 0% to 70%, 0% to 60%, 0% to 50%, 0% to 40%, 0% to 30%, 0% to 20%, 0% to 10%, or 0% to 5% of the first volume. The second volume can also be a non-zero level of volume.

After block 416, the controller circuit can reevaluate the noise profile relative to the noise profile threshold. In at least one embodiment, the controller circuit can increase the volume output level after a period of time. For example, the controller circuit can reduce the volume output level in response to the noise profile threshold being met, and when the noise profile threshold is not met, return the volume output level to the first volume. In at least one embodiment, the controller circuit can also increase the volume output level from the second volume to the first volume based on a time condition. The time condition can be based on a period of time to return to a first volume. For example, the controller circuit (after reducing the volume to the second volume) can automatically return to the first volume after at least 50 milliseconds have elapsed. In at least one embodiment, the time condition can be further conditioned on the noise profile threshold not being met. For example, the controller circuit can return to the first volume after at least 50 milliseconds have elapsed AND the noise profile threshold not being met.

In at least one embodiment, the controller circuit can ramp up (i.e., increase the volume gradually over time as opposed to a binary increase) to the first volume at a particular rate (i.e., a ramp rate). The ramp rate can be based on a time scale and a percentage of the first volume. In at least one embodiment, the ramp rate can include a third volume that is between the first and second volume after a period of time. For example, at time zero, the volume can be 5% of the first volume (e.g., the second volume); at time one, the volume can be 20% of the first volume (e.g., a third volume); at time two, the volume can be 40% of the first volume; at time three (e.g., a fourth volume), the volume can be 60% of the first volume; at time four, the volume can be 100% of the first volume. Thus, the volume can be returned incrementally over a time period. In at least one embodiment, the ramp rate can increase from 0 to 100% of the first volume during a time period from 50 to 2000 ms, 50 to 1000 ms, 50 to 500 ms, or 50 ms to 100 ms. [symbol] he controller circuit can also add an additional delay period, after determining the noise [symbol] reshold has not been met, before returning the volume to the first level (in a ramp up or instant adjustment). The delay period can be 50 to 1000 ms, for example. The delay period provides a window of time to ensure that the unwanted condition has concluded. This delay period can prevent unwanted changes in the volume before the unwanted condition has concluded.

In block 418, the controller circuit output the first volume based on the noise profile threshold not being met. For example, if there are no readings from the sensors that would indicate that an undesirable sound is likely to be amplified, then the controller circuit can maintain the volume level that is present by the user.

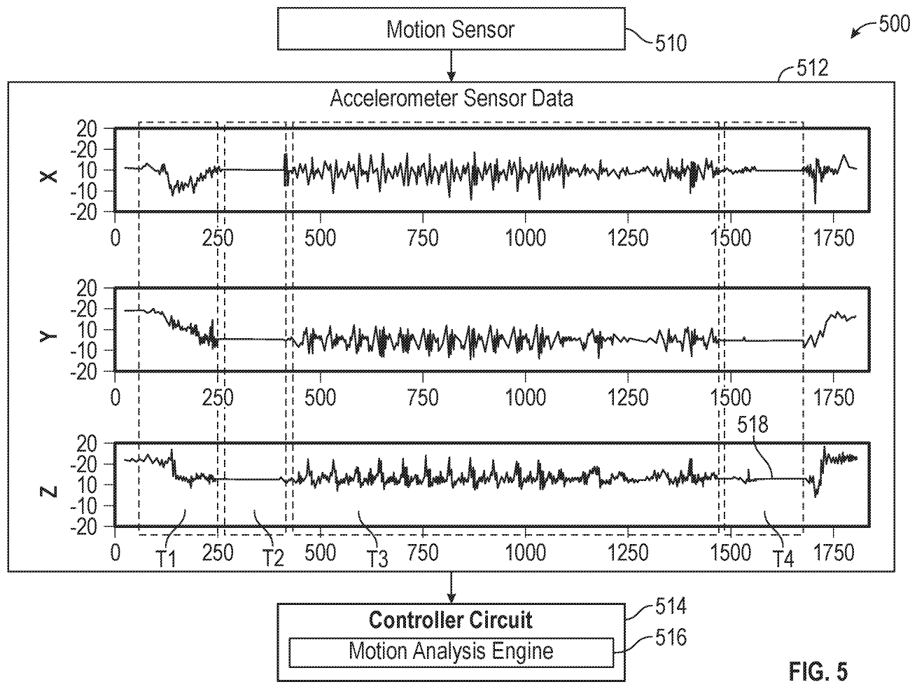

FIG. 5 illustrates a diagram 500 of determining a motion score from the plurality of sensor readings. The motion score can be useful in a noise profile. The motion score can be based on a motion sensor 510. For example, the motion sensor 510 can receive data relating to the motion of the stethoscope chestpiece. In at least one embodiment, the motion score can be based on the presence of motion (as illustrated). The motion score can also be based on the absence of motion. As shown, this motion can be along 3-axes (the X, Y, and Z axis).

As an illustrative example, the sample data 512 is captured by the motion sensor 510 and analyzed by the controller circuit 514. In at least one embodiment, the controller circuit 514 can have a dedicated engine (with optional dedicated processing resources), the motion analysis engine 516, to score the data from the motion sensor 510. Actual numerical scores can vary greatly depending on the metrics used, thus, in the following example, numerical scores are not provided, and only relative scores (scores relative to other time periods) are discussed herein.

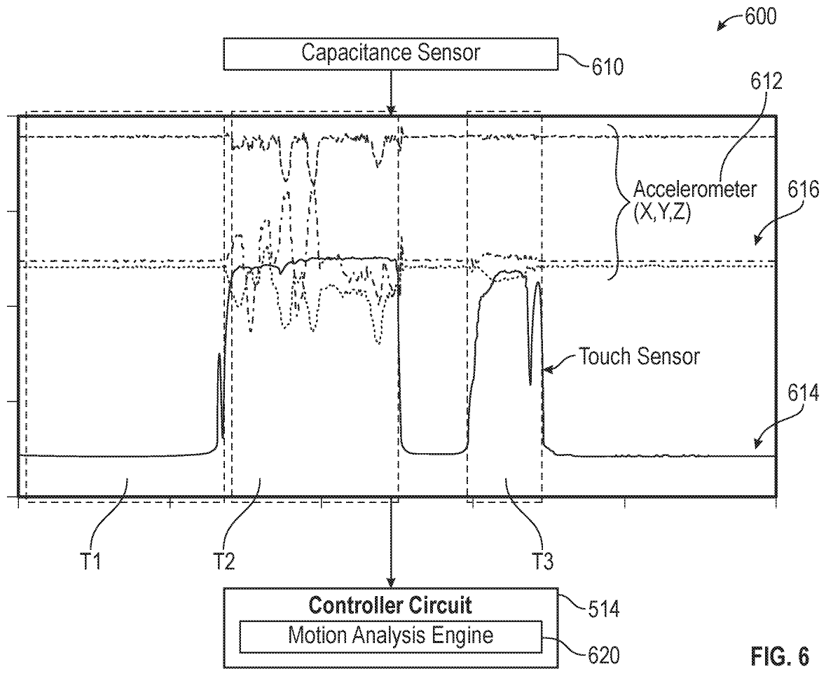

At T1, the motion sensor 510 receives motion signals that indicates a change in motion. For example, the data in the X-axis indicates a change and unstable chestpiece. At T2, the motion sensor 510 receives data that indicates a stable surface since there is little motion in any of the axes. Thus, the motion analysis engine 516 can score the period of T1 different from the period of T2 to indicate the lower motion of the chestpiece at T2. In at least one embodiment, the scoring can be performed at real time and per each sensor reading (i.e., the change of the reading relative to the immediate preceding value). The scoring can also be performed over a rolling average of sensor readings (as shown motion signals 616 in FIG. 6 (where the change in position (e.g. motion) for each axis (e.g. x,y,z) is filtered before threshold analysis) which can improve performance of the processor of the controller circuit 514. At T3, the motion data indicates that the chestpiece is moving and can be scored to indicate greater chestpiece motion than T1 or T2. At T4, the motion data indicates minimal motion but a slight increase of motion at time 518. Overall, the motion scores can indicate the chestpiece motion during the periods as follows (in order of higher chestpiece motion) [symbol] 4>T2. Thus, the motion analysis engine 516 can be responsive to the motion of chestpiece [symbol] or more axes.

In at least one embodiment, the motion can be scored based on a relationship of the motion relative to a motion threshold. For example, if the threshold is indicated by a variance of no greater than 10, then any motion value above 20 would obtain a higher score. The motion of the chestpiece can form at least part of the noise profile as described herein.

In at least one embodiment, the motion analysis engine 516 can also determine whether the motion meets a motion threshold. For example, the motion analysis engine 516 can determine that motion along periods T2 and T4 would meet not a noise profile threshold since they indicate periods of low motion of the chestpiece while motion along periods T1 and T3 would meet the noise profile threshold. If met, then the controller circuit accounts for the motion in the noise profile.

FIG. 6 illustrates a diagram 600 of an example analysis of the capacitance of a chestpiece. The analysis can include determining a capacitance score based on capacitance signals received from the capacitance sensor 610. As described, the capacitance sensor 610 can be electrically coupled to a portion of the chestpiece. The capacitance sensor 610 can measure electrical capacitance.

Sample data 612 is shown with capacitance signals 614 from the capacitance sensor superimposed over motion signals 616 from a motion sensor. The sample data 612 can be analyzed by the controller circuit 514. In at least one embodiment, the controller circuit 514 can have a dedicated engine (with optional dedicated processing resources), the capacitance analysis engine 620, to score the data from the capacitance sensor 610. In at least one embodiment, the capacitance analysis can be performed independently or in conjunction with one or more other analysis (e.g., motion analysis).

In at least one embodiment, the capacitance analysis engine 620 can determine a change in the capacitance signal that forms part of the noise profile. The capacitance analysis engine 620 can determine the change by comparing different capacitances obtained at different times or over time periods. For example, a rolling average can be used or the capacitance analysis engine 620 can determine variance of capacitance signals over a time period.

Actual numerical scores can vary greatly depending on the metrics used, thus, in the following example, numerical scores are not provided, and only relative scores (scores relative to other time periods) are discussed.

In at least one embodiment, the capacitance signal 614 can be binary. For example, the capacitance signal can be either on or off. If off, then there is no contact between the chestpiece and the fingers of a user. If on, then there is contact. In at least one embodiment, an unstable capacitance [symbol] 4 can indicate a loose finger grip or the user is adjusting/changing his/her grip on the [symbol] pe.

At T1, there is minimal change in capacitance and a low capacitance value which indicates that there is no contact between the chestpiece and a user's fingers. Period T1 also corresponds to low motion from a motion sensor and the user is not touching the chestpiece. At T2, the capacitance signal is stronger than at T1 and variance of the capacitance levels can indicate a stable finger grip position by a user. At T2, there is also a change in motion from the motion sensor (stethoscope is being held in the air . . . therefore there is x,y,z wobble). At T3, the capacitance signal is varying, which indicates that the finger grip position is unstable. However, the motion sensor at T3 indicates that there is little change in motion.

Thus, the capacitance analysis engine 620 can determine that the chestpiece is not held at T1. At T2, the capacitance signal indicates that the chestpiece is held in a stable position which would likely result in less noise contribution to the noise profile. At T3, the capacitance signal indicates that that chestpiece is not held with a stable hand grip. Thus, the degree of contribution to overall noise (e.g., in the noise profile) of the time periods would be ranked T3>T2>T1.

In at least one embodiment, the change in the capacitance signal can be scored. The capacitance score can be based on a relationship of the change in electrical capacitance (i.e., the capacitance signal) relative to the a capacitance change threshold. For example, the capacitance change at period T3 can indicate a variable capacitance signal compared to the change at period T2. Thus, the capacitance score can be higher for T3 relative to T2.

As illustrated, the noise profile can also include the motion of the chestpiece as shown by the motion sensor. When capacitance signals from T3 are analyzed contemporaneously with the motion signals, then controller circuit 514 can determine that the stability of the motion sensor can offset the unstable finger grip position and result in a determination of a lower noise contribution in the noise profile.

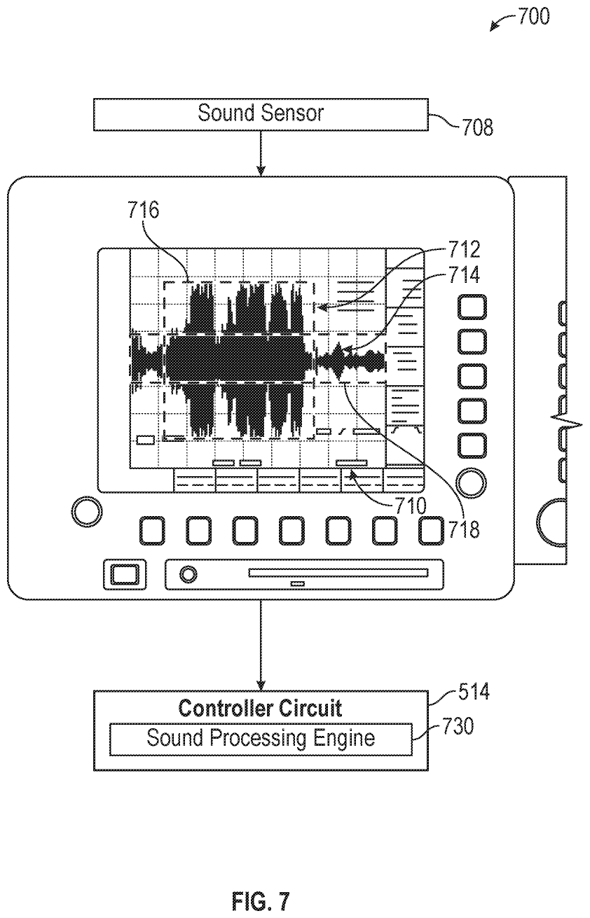

FIG. 7 illustrates a diagram 700 of an example analysis of the sound captured from a chestpiece. The diagram 700 shows a sound sensor 708. The sound sensor 708 can be positioned to monitor vibrations from a diaphragm on the chestpiece (on the side opposite the patient-facing side) as described herein. The sound sensor 708 can be communicatively coupled to the controller circuit 514. In at least one embodiment, a sound processing engine 730 can be configured to analyze the sound to determine if the sound is a drag sound.

For example, the sound sensor 708 can produce a phonocardiogram 710. The phonocardiogram 710 can be produced by the sound sensor 708 and the sound processing engine 730. [symbol] ocardiogram 710 is representative of the sound from the stethoscope. The phonocardiogram [symbol] rates the intensity of the sound versus the time. This can include noise sounds 712 and heart (or more broadly, auscultation) sounds 714. The heart sounds 714 can be distinguished from noise sounds 712 based on intensity and power of the sound signals and the relative frequency content. For example, the intensity of a heart sound 714 can be relatively lower and fall within the upper and lower boundaries of region 718. In at least one embodiment, any sound that has an intensity outside of the upper and lower boundaries of region 718 can be considered noise sound 712. The noise sound 712 can result from unintended contact caused by unintended motion by the stethoscope with another surface. For example, the noise sound 712 can be caused by the dragging of the stethoscope across clothing of the patient or the accidental dropping of the stethoscope across a hard surface. As shown herein, the noise sound intensity can remain within the upper and lower boundaries defined by region 716. In at least one embodiment, multiple regions of intensities can exist. For example, although two regions 716, 718 of intensities are shown, two or more regions of intensities with different upper and lower boundaries can be used to classify sound types and different types of noise (drag sound, tapping sound, voices). In at least one embodiment, the noise sound is indicative of a stethoscope diaphragm dragging against a surface in a coplanar manner. Heart sounds can be periodic and have minimal acoustic power over time (e.g. integrate signal). Consequently, the real-time “raw” audio signal may exceed a “simple threshold” at different times but is cannot be sustained over time (otherwise this is noise). Measuring the power of the acoustic signal over time is a useful sensor.

In addition, the frequency content of the acoustic sensor can the used to determine if the sound is non-biological. For example, heart sounds have a frequency range of 20-700 Hz, and lung sounds from 100-1500 Hz. Dragging, or moving, the stethoscope can generate significant acoustic power that can be sustained over time and contains a frequency profile that exceeds normal biological sounds of interest.

In at least one embodiment, the sound processing engine 730 can determine whether the auscultation sound corresponds to a noise sound based on an intensity and duration of the auscultation sound. In at least one embodiment, the sound processing engine 730 can also determine whether the sound corresponds to a noise sound based on the frequency of the sound. The controller circuit 514 can determine whether the presence of the noise sound forms part of the noise profile. The controller circuit 514 can determine the noise analysis before or in parallel with any electronic filtering. For example, electronic filters can filter out any sounds outside of a frequency band. The noise analysis can take place before electronic filtering.

In at least one embodiment, the noise sound can be scored. The noise sound score can be based on a relationship of the noise sound relative to a noise sound threshold. For example, a higher noise sound score can be related to the intensity of the sound. For example, if the intensity of the [symbol] above a boundary of a region (e.g., above the intensity of the sound in 718), then the degree [symbol] the intensity is above the boundary can be related to the noise sound score.

The noise sound score can be compared to a noise sound threshold. For example, if the noise sound intensity is below the boundaries of region 716, then the noise sound may not be included in the noise profile and thus, not classified as noise.

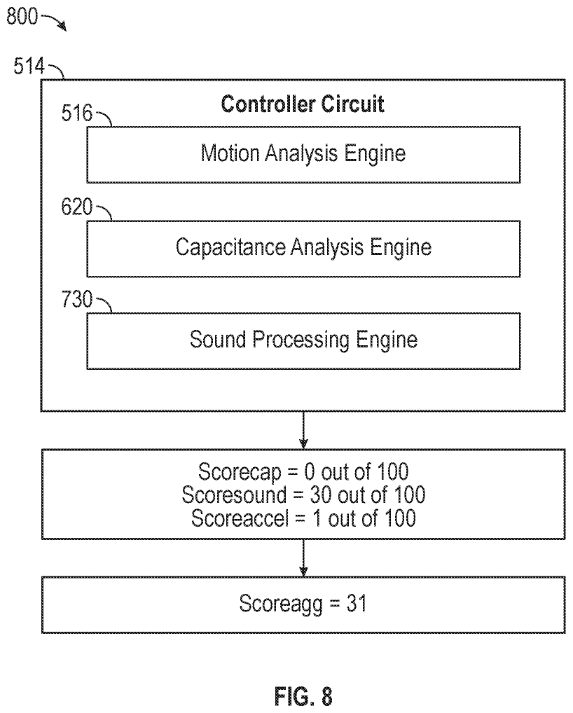

FIG. 8 illustrates a flow diagram 800 of an example of scoring multiple inputs from different sensors. The controller circuit 514, including the motion analysis engine 516, the capacitance analysis engine 620, and the sound processing engine 730 can produce a motion score, a capacitance score, and a noise sound score, respectively.

If the motion score is 1 out of 100, the capacitance score is 0 out of 100, and the noise sound score is 30 out of 100, then the controller circuit 514 can aggregate the score to form the aggregate score 31. The aggregate score 31 can form a noise profile score. The noise profile score can be part of the noise profile. The aggregate score can account for the scores of the inputs that can result in noise for the user. In at least one embodiment, one or more of the sensor scores can be weighted. For example, the if the motion score has a higher weight than the noise sound score, then the aggregate score can be higher than 31.

In at least one embodiment, the controller circuit 514 can compare the resulting noise profile score with the noise profile threshold. If the noise profile threshold is met, then the volume of the speakers of the stethoscope can be reduced as described herein.

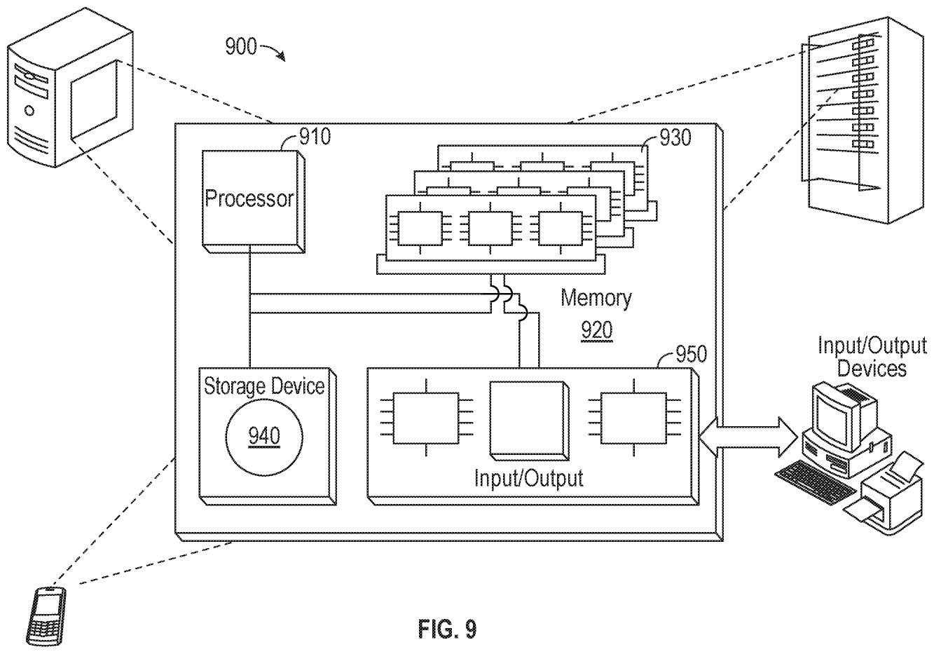

FIG. 9 shows a detailed example of various devices that may be configured to execute program code to practice some examples in accordance with the current disclosure. The controller circuit 900 can be an embodiment of controller circuit 112 or 514 as used herein. For example, controller circuit 900 may be a computing device that performs any of the techniques described herein. In the example, a controller circuit 900 includes a processor 910 that is operable to execute program instructions or software, causing the computer to perform various methods or tasks. Processor 910 is coupled via bus 920 to a memory 930, which is used to store information such as program instructions and other data while the computer is in operation. A storage device 940, such as a hard disk drive, nonvolatile memory, or other non-transient storage device stores information such as program instructions, data files of the multidimensional data and the reduced data set, and other information. The controller circuit 900 may also include various input-output elements 950, including parallel or serial ports, USB, Firewire or IEEE 1394, Ethernet, and other such ports to connect the computer to external device such as a printer, video camera, surveillance equipment or the like. Other input-output elements may include wireless communication interfaces such as Bluetooth, Wi-Fi, and [symbol] ata networks. In at least one embodiment, FIG. 9 can also illustrate the general layout of the [symbol] cuit 114 from FIG. 1.

### LIST OF ILLUSTRATIVE EMBODIMENTS

1. A stethoscope, comprising:

a chestpiece, having an inside surface and an outside surface, a portion of the inside surface forms a bell and a portion of the outside surface is electrically conductive;

a plurality of sensors, including a first sensor, and a second sensor;

a speaker communicatively coupled to the first sensor;

a controller circuit that comprises one or more computer processors communicatively coupled to a memory, the memory comprising instructions that when executed by the one or more computer processors cause the one or more computer processors to:

receive a plurality of sensor readings from the plurality of sensors;

determine a noise profile based on the plurality of sensor readings and a first volume output through the speaker;

determine whether a noise profile threshold is met by the noise profile;

reducing volume output through the speaker from the first volume to a second volume based on the noise profile threshold being met.

2. The stethoscope of embodiment 1, further comprising a diaphragm coupled to the chestpiece having a first side and a second side, the second side faces the inside surface of the chestpiece; wherein the first sensor is positioned to monitor vibrations from the diaphragm on the second side, and the controller circuit comprises memory comprising instructions that when executed by the one or more computer processors cause the one or more computer processors to:

receive an indication corresponding to a sound from the first sensor;

determine whether the sound corresponds to a noise sound based on an intensity and duration of the sound, wherein presence of the noise sound forms part of the noise profile.

3. The stethoscope of embodiment 2, wherein the noise sound includes a drag sound indicative of a stethoscope diaphragm dragging against a surface in a coplanar manner.
4. The stethoscope of any of embodiments 1 or 3, wherein the memory comprises instructions that when executed by the one or more computer processors cause the one or more computer processors to:

perform signal processing on an auscultation sound from the first sensors to produce a processed signal; and

output the processed signal to the speaker at the first volume. [symbol] ethoscope of any of embodiments 1 to 4, wherein the second sensor is a capacitance sensor electrically coupled to a conductive portion of the outside surface,

wherein the memory comprises instructions that when executed by the one or more computer processors cause the one or more computer processors to:
receive a capacitance signal from the second sensor,
determine a change in the capacitance signal, wherein the change in the capacitance signal forms part of the noise profile.
   6. The stethoscope of embodiment 5, wherein the memory comprises instructions that when executed by the one or more computer processors cause the one or more computer processors to:

determine whether the capacitance change meets an capacitance threshold,

if met, then provide whether the capacitance change threshold is met to the noise profile.

7. The stethoscope of any of embodiments 1 to 6, wherein the second sensor is a motion sensor proximate to the chestpiece such that motion of the chestpiece in at least a two-dimensional space is obtained;

wherein the memory comprises instructions that when executed by the one or more computer processors cause the one or more computer processors to:
receive a signal from the third sensor in response to motion of the chestpiece along one or more axes, wherein the motion forms at least part of the noise profile.
   8. The stethoscope of embodiment 7, wherein the memory comprises instructions that when executed by the one or more computer processors cause the one or more computer processors to:

determine whether the motion meets a motion threshold;

if met, then provide the motion threshold being met into the noise profile.

8a. The stethoscope of any of embodiments 1 to 8, further comprising a third sensor, wherein the third sensor is a motion sensor, a capacitance sensor, proximity sensor, or combination thereof.
8b. The stethoscope of any of embodiments 1 to 8, wherein the second sensor is a sound sensor, motion sensor, a capacitance sensor, proximity sensor, or combination thereof.
8c. The stethoscope of any of embodiments 1 to 8, wherein the first sensor and second sensor are different. [symbol] tethoscope of any of embodiments 1 to 8, wherein the first sensor is a sound sensor, motion [symbol] capacitance sensor, proximity sensor, or combination thereof.
9. The stethoscope of any of embodiments 1 to 8, wherein reducing the volume output occurs no greater than 50 ms from the first volume being output through the speaker.
10. The stethoscope of any of embodiments 1 to 9, wherein a duration of the second volume occurs for as long as the noise profile threshold is met.
11. The stethoscope of any of embodiments 1 to 10, the memory comprising instructions that when executed by the one or more computer processors cause the one or more computer processors to:

output the first volume based on the noise profile threshold not being met.

12. The stethoscope of any of embodiments 1 to 10, wherein the output to the first volume is ramped at a ramp rate.
13. The stethoscope of embodiment 12, wherein the ramp rate is from 0 to 100% of the first volume in 50 to 500 ms.
13a. The stethoscope of any of embodiments 1 to 13, wherein the output the first volume comprises delaying increasing to the first volume from the second volume by at least 500 ms.
14. The stethoscope of any of embodiments 1 to 13, wherein the memory comprising instructions that when executed by the one or more computer processors cause the one or more computer processors to:

output at a third volume that is between the first and second volume after a period of time.

15. The stethoscope of any of embodiments 1 to 14, wherein the memory comprises instructions that when executed by the one or more computer processors cause the one or more computer processors to determine a noise profile based on the plurality of sensor readings by:
scoring the noise sound based on a relationship of an auscultation sound relative to the noise sound threshold.
   16. The stethoscope of any of embodiments 1 to 15, wherein the memory comprises instructions that when executed by the one or more computer processors cause the one or more computer processors to determine a noise profile based on the plurality of sensor readings by: [symbol] scoring the change in the capacitance signal based on a relationship of the change [symbol] elative to the capacitance change threshold.
   17. The stethoscope of any of embodiments 1 to 16, wherein the memory comprises instructions that when executed by the one or more computer processors cause the one or more computer processors to determine a noise profile based on the plurality of sensor readings by:

scoring the motion based on a relationship of the motion relative to the motion threshold.

18. The stethoscope of any of embodiments 1 to 17, wherein the memory comprises instructions that when executed by the one or more computer processors cause the one or more computer processors to determine a noise profile based on the plurality of sensor readings by:

aggregating the noise sound score, the change in capacitance score, the motion score, or combinations thereof to form a noise profile score;

comparing the noise profile score with the noise profile threshold.

19. The stethoscope of any of embodiments 1 to 18, further comprising a yoke, and tubing, wherein the ear tips are fluidically coupled to the speaker through the yoke and tubing.
20. The stethoscope of embodiment 19, wherein the speaker is separate from the chestpiece.
21. The stethoscope of any of embodiments 1 to 20, further comprising a proximity sensor disposed proximate to the diaphragm; wherein the memory comprises instructions that when executed by the one or more computer processors cause the one or more computer processors to determine whether a diaphragm contacts a patient based on the proximity sensor; and reduce volume output to the second volume, in response to the diaphragm not being in contact with the patient.
22. The stethoscope of any of embodiments 1 to 21, wherein the first sensor is a piezoelectric microphone directly adhered to the diaphragm with an adhesive.
23. A method of reducing volume in an electronic stethoscope, comprising:
a chestpiece, having an inside surface and an outside surface, a portion of the inside surface forms a bell and a portion of the outside surface is electrically conductive;

receive a plurality of sensor readings from a plurality of sensors including a first sensor, and a second sensor;

determine, with a controller circuit, a noise profile based on the plurality of sensor readings and a first volume output through a speaker communicatively coupled to the first sensor;

determine whether a noise profile threshold is met by the noise profile; [symbol] educing volume output through the speaker from the first volume to a second volume based [symbol] ise profile threshold being met.

24. The method of embodiment 23, further comprising:

receiving an indication corresponding to a sound from the first sensor;

determine whether the sound corresponds to a noise sound based on an intensity and duration of the sound, wherein presence of the noise sound forms part of the noise profile.

25. The method of embodiment 24, wherein the noise sound includes a drag sound indicative of a stethoscope diaphragm dragging against a surface in a coplanar manner.
26. The method of any of embodiments 23 to 25, further comprising:

performing signal processing on an auscultation sound from the first sensors to produce a processed signal; and

outputting the processed signal to the speaker at the first volume.

27. The method of any of embodiments 23 to 26, further comprising:
receiving a capacitance signal from the second sensor,
determining a change in the capacitance signal, wherein the change in the capacitance signal forms part of the noise profile.
   28. The method of embodiment 27, further comprising:

determining whether the capacitance change meets an capacitance threshold,

if met, then providing whether the capacitance change threshold is met to the noise profile.

29. The method of any of embodiments 23 to 28, further comprising:
receiving a signal from the third sensor in response to motion of the chestpiece along one or more axes, wherein the motion forms at least part of the noise profile.
   30. The method of embodiment 29, further comprising:

determining whether the motion meets a motion threshold;

if met, then providing the motion threshold being met into the noise profile.

31. The method of any of embodiments 23 to 30, wherein reducing the volume output occurs no greater than 50 ms from the first volume being output through the speaker. [symbol] nethod of any of embodiments 23 to 31, wherein a duration of the second volume occurs for [symbol] s the noise profile threshold is met.
33. The method of any of embodiments 23 to 32, further comprising:

outputting sound through the speaker at the first volume based on the noise profile threshold not being met.

34. The method of embodiment 33, further comprising ramping the second volume to the first volume at a ramp rate.
35. The method of any of embodiments 33 or 34, wherein the outputting the first volume comprises delaying increasing to the first volume from the second volume by at least 500 ms.
36. The method of any of embodiments 23 to 35, further comprising outputting sound at a third volume that is between the first and second volume after a period of time.
37. The method of any of embodiments 23 to 36, further comprising scoring the noise sound based on a relationship of an auscultation sound relative to the noise sound threshold.
38. The method of any of embodiments 23 to 37, further comprising scoring the change in the capacitance signal based on a relationship of the change relative to the capacitance change threshold.
39. The method of any of embodiments 23 to 38, further comprising scoring the motion based on a relationship of the motion relative to the motion threshold.
40. The method of any of embodiments 23 to 40, further comprising:

aggregating the noise sound score, the change in capacitance score, the motion score, or combinations thereof to form a noise profile score;

comparing the noise profile score with the noise profile threshold.

41. The method of any of embodiments 23 to 40, further comprising:

determining that the noise profile threshold is met by the noise profile based on a proximity sensor indicating that a diaphragm contacts a patient.

42. An electronic stethoscope comprising: [symbol] chestpiece, having an inside surface and an outside surface, a portion of the inside surface) [symbol] ell and a portion of the outside surface is electrically conductive;

a speaker communicatively coupled to a first sensor, wherein the first sensor is a piezoelectric microphone;

a diaphragm coupled to the chestpiece having a first side and a second side, the second side faces the inside surface of the chestpiece, wherein the first sensor is positioned to monitor vibrations from the diaphragm on the second side, wherein the piezoelectric microphone is directly adhered to the diaphragm with an adhesive; further comprising

a controller circuit that comprises one or more computer processors communicatively coupled to a memory, the memory comprising instructions that when executed by the one or more computer processors cause the one or more computer processors to process sounds from the first sensor and output the sounds through the speaker.

43. The electronic stethoscope of embodiment 42, wherein the electronic stethoscope is configured to perform the method of any of embodiments 23 to 41.

1. A stethoscope, comprising:
a chestpiece, having an inside surface and an outside surface, a portion of the inside surface forms a bell and a portion of the outside surface is electrically conductive;
a plurality of sensors, including a first sensor, and a second sensor;
a speaker communicatively coupled to the first sensor;
a controller circuit that comprises one or more computer processors communicatively coupled to a memory, the memory comprising instructions that when executed by the one or more computer processors cause the one or more computer processors to:
receive a plurality of sensor readings from the plurality of sensors;
determine a noise profile based on the plurality of sensor readings and a first volume output through the speaker;
determine whether a noise profile threshold is met by the noise profile; and
reduce volume output through the speaker from the first volume to a second volume based on the noise profile threshold being met.

2. The stethoscope of claim 1, further comprising a diaphragm coupled to the chestpiece having a first side and a second side, the second side faces the inside surface of the chestpiece; wherein the first sensor is positioned to monitor vibrations from the diaphragm on the second side, and the controller circuit comprises memory comprising instructions that when executed by the one or more computer processors cause the one or more computer processors to:
receive an indication corresponding to a sound from the first sensor;
determine whether the sound corresponds to a noise sound based on an intensity and duration of the sound, wherein presence of the noise sound forms part of the noise profile.

3. The stethoscope of claim 2, wherein the noise sound includes a drag sound indicative of a stethoscope diaphragm dragging against a surface in a coplanar manner.

4. The stethoscope of claim 1, wherein the memory comprises instructions that when executed by the one or more computer processors cause the one or more computer processors to:
perform signal processing on an auscultation sound from the first sensors to produce a processed signal; and
output the processed signal to the speaker at the first volume.

5. The stethoscope of claim 1, wherein the second sensor is a capacitance sensor electrically coupled to a conductive portion of the outside surface,
wherein the memory comprises instructions that when executed by the one or more computer processors cause the one or more computer processors to:
receive a capacitance signal from the second sensor,
determine a change in the capacitance signal, wherein the change in the capacitance signal forms part of the noise profile.

6. The stethoscope of claim 5, wherein the memory comprises instructions that when executed by the one or more computer processors cause the one or more computer processors to:
determine whether the capacitance change meets an capacitance threshold,
if met, then provide whether the capacitance change threshold is met to the noise profile.

7. The stethoscope of claim 1, wherein the second sensor is a motion sensor proximate to the chestpiece such that motion of the chestpiece in at least a two-dimensional space is obtained;
wherein the memory comprises instructions that when executed by the one or more computer processors cause the one or more computer processors to:
receive a signal from the third sensor in response to motion of the chestpiece along one or more axes, wherein the motion forms at least part of the noise profile.

8. The stethoscope of claim 7, wherein the memory comprises instructions that when executed by the one or more computer processors cause the one or more computer processors to:
determine whether the motion meets a motion threshold;
if met, then provide the motion threshold being met into the noise profile.

9. The stethoscope of claim 1, wherein reducing the volume output occurs no greater than 50 ms from the first volume being output through the speaker.

10. The stethoscope of claim 1, wherein a duration of the second volume occurs for as long as the noise profile threshold is met.

11. The stethoscope of claim 1, the memory comprising instructions that when executed by the one or more computer processors cause the one or more computer processors to:
output the first volume based on the noise profile threshold not being met.

12. The stethoscope of claim 1, wherein the output to the first volume is ramped at a ramp rate.

13. The stethoscope of claim 1, wherein the output the first volume comprises delaying increasing to the first volume from the second volume by at least 500 ms.

14. The stethoscope of claim 1, further comprising a proximity sensor disposed proximate to the diaphragm; wherein the memory comprises instructions that when executed by the one or more computer processors cause the one or more computer processors to determine whether a diaphragm contacts a patient based on signals from the proximity sensor; and reduce volume output to the second volume, in response to the diaphragm not being in contact with the patient.

15. The stethoscope of claim 1, wherein the first sensor is a piezoelectric microphone directly adhered to the diaphragm with an adhesive.

16. A method of reducing volume in an electronic stethoscope, comprising:
a chestpiece, having an inside surface and an outside surface, a portion of the inside surface forms a bell and a portion of the outside surface is electrically conductive;
receive a plurality of sensor readings from a plurality of sensors including a first sensor, and a second sensor;
determine, with a controller circuit, a noise profile based on the plurality of sensor readings and a first volume output through a speaker communicatively coupled to the first sensor;
determine whether a noise profile threshold is met by the noise profile;
reducing volume output through the speaker from the first volume to a second volume based on the noise profile threshold being met.

17. The method of claim 16, further comprising:
receiving an indication corresponding to a sound from the first sensor;
determine whether the sound corresponds to a noise sound based on an intensity and duration of the sound, wherein presence of the noise sound forms part of the noise profile.

18. The method of claim 16, further comprising:
performing signal processing on an auscultation sound from the first sensors to produce a processed signal; and
outputting the processed signal to the speaker at the first volume.

19. The method of claim 16, further comprising:
receiving a capacitance signal from the second sensor,
determining a change in the capacitance signal, wherein the change in the capacitance signal forms part of the noise profile.

20. The method of claim 16, further comprising:
receiving a motion signal in response to motion of the chestpiece along one or more axes, wherein the motion signal forms at least part of the noise profile.
